(19) United States
(12) Patent Application Publication
(10) Pub. No.: US 2025/0025704 A1
(43) Pub. Date: Jan. 23, 2025

(54) ELEVATOR DEFIBRILLATOR SYSTEM

(71) Applicant: GOOD ELEVATOR CO., LTD., Busan (KR)

(72) Inventor: Kwon Jin JE, Busan (KR)

(21) Appl. No.: 18/711,066

(22) PCT Filed: Mar. 23, 2023
(86) PCT No.: PCT/KR2023/003834

§ 371 Date: Oct. 10, 2024

(30) Foreign Application Priority Data
Mar. 24, 2022 (KR) 10-2022-0036406

(51) Int. Cl. A61N 1/39; B66B 1/24; B66B 1/34

(52) U.S. Cl. CPC A61N 1/3904; A61N 1/3925; B66B 1/24; B66B 1/34

(57) ABSTRACT

The present invention relates to an elevator defibrillator system in which a defibrillator, which is an automated external defibrillator, is installed inside an elevator, and a controller of the defibrillator is synchronously controlled with an elevator control panel. In addition, when the defibrillator is operated, the elevator control panel stops the driving function and floor stops and allows operation only to the first floor. Also, a CCTV camera is installed inside the elevator or inside a defibrillator storage case. Furthermore, when the defibrillator is operated, a control unit transmits a signal to a server of a defibrillator management institution, and the present invention has the remarkable effects of enabling safe use of the defibrillator, and increasing the probability of saving a life by rapidly reaching the ground by means of synchronous-control with the elevator. Moreover, when the defibrillator is operated, a related institution is automatically contacted and necessary measures are provided, thereby having a remarkable effect of saving a patient by facilitating accurate operation the defibrillator. Besides, a defibrillator case may be used as a footrest, or the like, and it is possible to open a cover with one hand and take out the defibrillator, and accordingly, the present invention has a remarkable effect of convenient operation.

### TECHNICAL FIELD

The present disclosure relates to an elevator defibrillator system and, more particularly, to an elevator defibrillator system enabling safe use of a defibrillator, increasing the chance of saving lives by means of synchronous-control with an elevator to enable the elevator to rapidly reach the ground, and of saving a patient by facilitating accurate operation of a defibrillator by automatically notifying a related institution and guiding a user to perform necessary measures when operating the defibrillator, providing a defibrillator case as a footrest, etc., and enabling convenient operation by enabling the user to open a cover with one hand and remove the defibrillator from the case.

### BACKGROUND ART

As the related art of a cardiac defibrillator storage box installed in an elevator, Korean Patent Application Publication No. 10-2015-0125221 discloses the cardiac defibrillator storage box installed in an inner surface of an elevator. However, the cardiac defibrillator storage case installed in an attached type in the inner surface of the elevator
reduces the size of the internal space of the elevator by protruding into the internal space, it is difficult to install the protruding-type cardiac defibrillator storage case in the interval space of an elevator, which is formed in accordance with space standards set by regulations.

As another related art, Korean Patent Application Publication No. 10-2013-0127565 discloses the technique for a cardiac defibrillator storage case, which is configured to be usually stored outside of a frame of an elevator and being removed downward through a hole of the ceiling of the elevator for use thereof. However,
the raising-and-lowering-type case has a problem that the use thereof requires the additional step of lowering same in an emergency situation.

Furthermore, Korean Patent No. 10-1616289 discloses the AED system of elevator, the system including: an AED switch provided next to an elevator opening door or on a home automation key input unit;
an elevator, which, when the AED switch transmits an AED key push signal, is moved to the relevant floor where the AED switch was pushed; and
an AED storage box mounted in the elevator, and accommodating and storing an AED (automated external defibrillator), and turning on LEDs, which are mounted to one portion thereof, when the AED key push signal is transmitted from the elevator,
wherein the AED storage box is stored in the ceiling of the elevator, the AED storage box being mounted above a portion beside the door in the ceiling of the elevator, and an LED display unit being provided at a lower surface of the AED storage box to be seeable from the internal space of the elevator when the AED storage box is stored in the ceiling of the elevator, and
the system further includes: a support rod mounted to an upper end of the AED storage box, and mounted with a spring intervening on an main path located inside the ceiling of the elevator; and an AED storage box handle mounted to a lower end of the AED storage box, and located on a side wall of the elevator.

However, the related arts have the disadvantage that use of a defibrillator is not safe, and the chance of saving lives is lowered since it is difficult for the elevator to quickly reach the ground due to interference by other users during moving to the ground.

Furthermore, there is also the problem that a precious life of a patient is lost when a user of the defibrillator operates the defibrillator incorrectly due to the difficulty in operating a defibrillator.

Furthermore, there is the disadvantage that it takes a lot of time or is inconvenient to remove the defibrillator from a defibrillator case.

### DISCLOSURE

### Technical Problem

Accordingly, the present disclosure has been made keeping in mind the above problems occurring in the related art, and the present disclosure is intended to provide an elevator defibrillator system having the remarkable effects of enabling safe use of a defibrillator and increasing of the chance of saving lives by means of synchronous-control with an elevator to enable the elevator to rapidly reach the ground.

Another objective of the present disclosure is to provide an elevator defibrillator system having the remarkable effect of saving a patient by facilitating accurate operation of a defibrillator by automatically notifying a related institution and guiding a user to perform necessary measures when operating the defibrillator.

Yet another objective of the present disclosure is to provide an elevator defibrillator system having the remarkable effects of providing the defibrillator case as a footrest, etc., and enabling convenient operation by enabling a user to open a cover with one hand and remove a defibrillator.

### Technical Solution

The present disclosure relates to an elevator defibrillator system in which a defibrillator, which is an automated cardiac defibrillator, is installed inside an elevator, and the elevator defibrillator system is characterized in that a defibrillator controller is synchronously controlled with an elevator control panel.

Furthermore, the elevator defibrillator system is characterized in that when the defibrillator is operated, an elevator control panel stops stopping and moving functions for other floors and allows the elevator to be moved only to the first floor.

Furthermore, the elevator defibrillator system is characterized in that a CCTV camera is installed inside the elevator or a defibrillator storage case.

Furthermore, the elevator defibrillator system is characterized in that when the defibrillator is operated, a control unit transmits a signal to a server of a defibrillator management institution.

### Advantageous Effects

Therefore, the elevator defibrillator system of the present disclosure has the remarkable effects of enabling safe use of the defibrillator and increasing the chances of saving lives by means of synchronous-control with the elevator to enable the elevator to rapidly reach the ground.

Furthermore, there is the remarkable effect of saving a patient by facilitating accurate operation the defibrillator by automatically notifying a related institution and guiding a user to perform necessary measures when operating the defibrillator.

Furthermore, there are the effects of using the defibrillator case as a footrest and enabling convenient operation for the user to open the cover with one hand and remove the defibrillator from the case.

### DESCRIPTION OF DRAWINGS

FIG. 1 is an example view showing an installation location of a wall-embedded defibrillator inside an elevator according to the present disclosure.

FIG. 2 is a view showing an installation state of the defibrillator view inside the elevator of the present disclosure.

FIG. 3 is a perspective view showing a quadrangular defibrillator storage case of the defibrillator inside the elevator of the present disclosure.

FIG. 4 is a perspective view showing a cylindrical defibrillator storage case of the defibrillator inside the elevator of the present disclosure.

FIG. 5 is a perspective view showing a fan-shaped defibrillator storage case of the defibrillator inside the elevator of the present disclosure.

### DESCRIPTION FOR REFERENCE NUMERALS

100: defibrillator
110: elevator frame 111: elevator door
120: defibrillator storage case 121: transparent resin cover

### BEST MODE

The present disclosure relates to an elevator defibrillator system in which a defibrillator, which is an automated cardiac defibrillator, is installed inside an elevator and a defibrillator controller is synchronously controlled with an elevator control panel.

Furthermore, when the defibrillator is operated, a control unit of the defibrillator transmits a signal to the elevator control panel and the elevator control panel stops stopping and moving functions at floors other than the first floor to temporarily limit usage by users and allow the elevator to be moved only to the first floor.

Furthermore, the controller and the control panel have respective communication modules to wirelessly communicate with each other. The controller includes a transmitting and receiving unit, a storage unit, and the control unit installed therein.

Furthermore, a power source of the defibrillator may be used by connecting the commercial power thereto and may be used by charging with a charged battery installed.

Furthermore, when the defibrillator is operated, the control unit notifies, through the transmitting unit, a smartphone of an apartment manager, a security office, 119, or an emergency management department call of an emergency situation by SNS. Furthermore, when the defibrillator is operated, the control unit notifies a server of a related hospital through the transmitting unit, and the hospital server notifies a smartphone of an attending specialist so that the attending specialist is on standby in a hospital emergency room.

Furthermore, when the defibrillator is operated, the control unit notifies the related hospital through the transmitting unit, and the hospital server notifies a smartphone of a driver of an ambulance, so that the ambulance will arrive at the building with the relevant elevator and be on standby.

Furthermore, operating the defibrillator is configured such that when the power source in the defibrillator is turned on, the communication module transmits a signal to the hospital server.

A CCTV camera may be installed inside the elevator or a defibrillator storage case.

Furthermore, the camera in the case is mainly attached to an inner surface of a cover, so that it facilitates user of the defibrillator by enabling the camera to be easily viewed by positioning the inner surface of the cover to face the user. Therefore, when the defibrillator is operated, the control unit transmits a signal to a server of a defibrillator management institution. When the defibrillator is operated and an operation signal of the defibrillator is received, a control unit server of a public health center, the disease control and prevention center, etc., which are the defibrillator management institutions, operates speakers installed inside the defibrillator case to enable the user to manually turn on the CCTV camera by voice.

Furthermore, when the defibrillator is operated, the controller controls the CCTV camera so that the CCTV camera is automatically operated, and the defibrillator management institution is equipped with a database containing user manuals, etc., about emergency measures in an emergency situation.

Furthermore, when the defibrillator cover is opened, a contact sensor transmits a signal to the control unit of the defibrillator controller and the control unit notifies the server of the defibrillator management institution of opening of the defibrillator cover and allows the CCTV camera attached to the inner surface of the cover to be automatically operated.

Furthermore, the server of the defibrillator management institution informs the user of the defibrillator about a method for using the defibrillator while observing situations inside the elevator with the CCTV camera attached to the defibrillator cover. In addition, the server of the defibrillator management institution wirelessly transmits methods to the defibrillator controller for using functions of specific measures depending on a patient, and the control unit loads a data for the usage method stored in the storage unit and displays the data so that the user of the defibrillator can operate the relevant function.

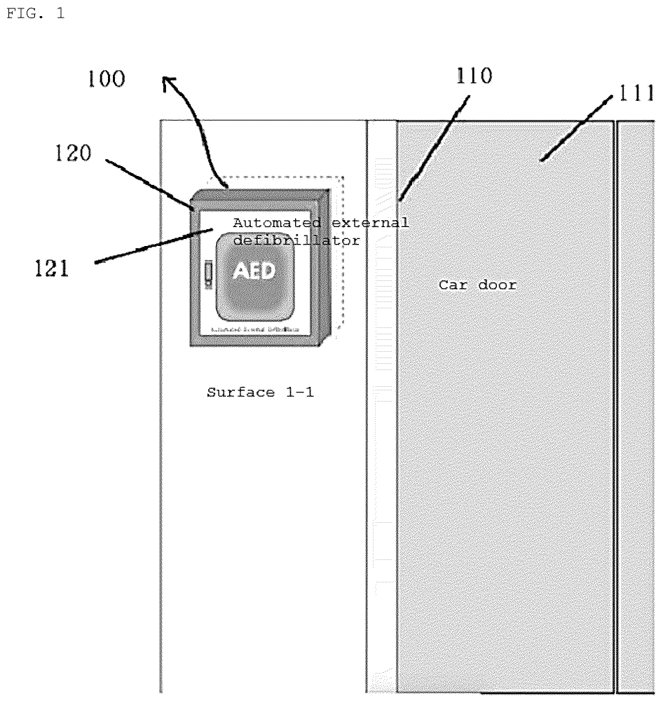

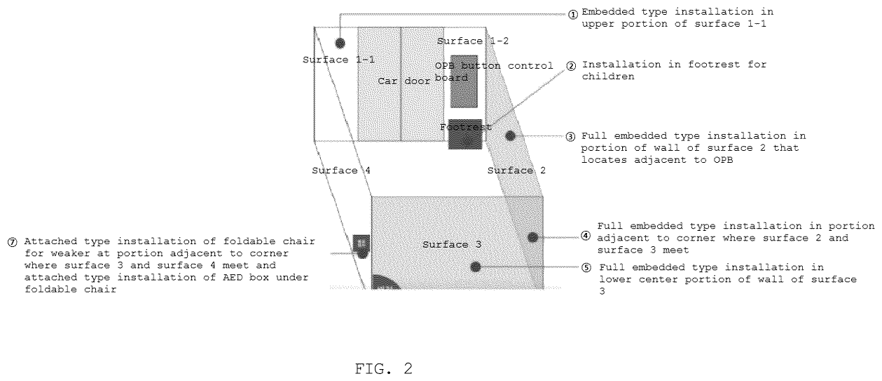

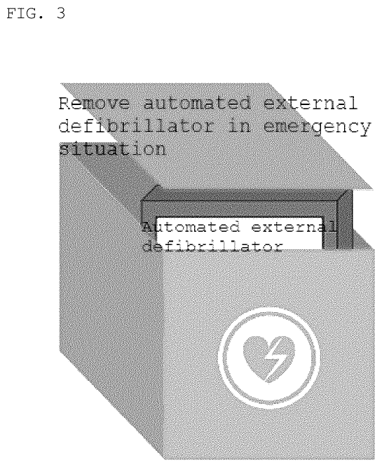

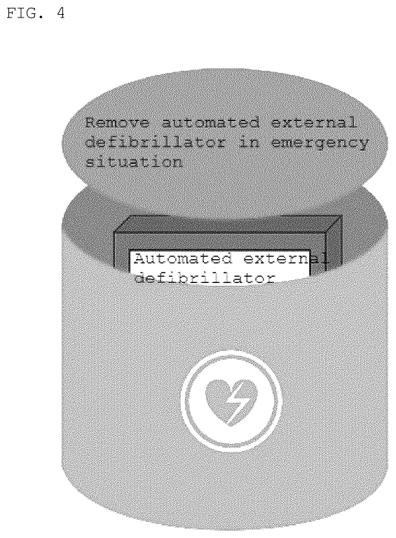

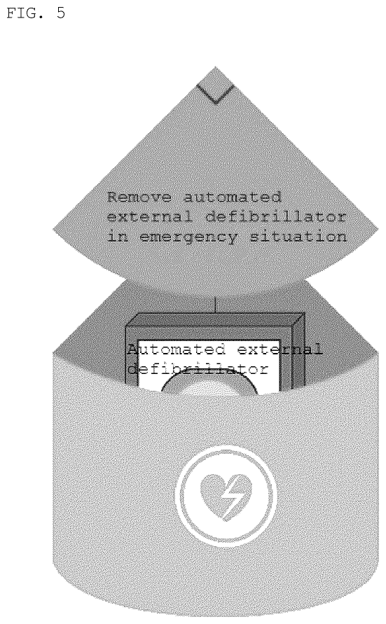

The present disclosure will be described in detail with reference accompanying drawings. FIG. 1 is an example view showing an installation location of a wall-embedded defibrillator inside an elevator according to the present disclosure. FIG. 2 is a view showing an installation state of the defibrillator view inside the elevator of the present disclosure. FIG. 3 is a perspective view showing a quadrangular defibrillator storage case of the defibrillator inside the elevator of the present disclosure. FIG. 4 is a perspective view showing a cylindrical defibrillator storage case of the defibrillator inside the elevator of the present disclosure. FIG. 5 is a perspective view showing a fan-shaped defibrillator storage case of the defibrillator inside the elevator of the present disclosure.

The elevator defibrillator system of the present disclosure includes a defibrillator, which is an automated cardiac defibrillator, installed in an elevator. Specifically, the defibrillator controller may be synchronously controlled with the elevator control panel.

The defibrillator is immediately operated, the control unit of the defibrillator transmits a signal to the elevator control panel, and the elevator control panel stops stopping and moving functions at floors other than the first floor to temporarily limit use by other users and allow the elevator to be moved only to the first floor.

The controller and the control panel have respective the communication modules to wirelessly communicate with each other, and the controller includes the transmitting and receiving unit, the storage unit, and the control unit installed therein.

The power source of the defibrillator may be used by connecting the commercial power thereto and may be used by charging with a charged battery installed.

Furthermore, according to the present disclosure, when the defibrillator is operated, the control unit notifies, through the transmitting unit, a smartphone of an apartment manager, a security office, 119, or an emergency management department call of an emergency situation by SNS. Furthermore, when the defibrillator is operated, the control unit notifies the server of a related hospital through the transmitting unit, and the hospital server notifies a smartphone of an attending specialist so that the attending specialist is on standby in a hospital emergency room.

When the defibrillator is operated, the control unit notifies the related hospital server through the transmitting unit, and the hospital server notifies a smartphone of a driver of an ambulance, so that the ambulance will arrive at the building with the relevant elevator and be on standby.

Operating the defibrillator is configured such that when the power source in the defibrillator is turned on, the communication module transmits a signal to the hospital server. Furthermore, according to the present disclosure, the CCTV camera may be installed inside the elevator or the defibrillator storage case.

The camera inside the case is attached to the inner surface of the cover to enable the user to easily face the camera when the inner surface of the cover is positioned to face the user of the defibrillator. Therefore, when the defibrillator is operated, the control unit transmits a signal to the server of the defibrillator management institution. When the defibrillator is operated and a defibrillator operation signal is received, a server control unit of, for example, a public health center or a disease control and prevention center, which is the defibrillator management institution, operates the speakers provided inside the defibrillator case to allow the user to manually turn on the CCTV camera by voice. On the other hand, when the defibrillator is operated, the controller controls the CCTV camera so that the CCTV camera is automatically operated. The defibrillator management institution is equipped with the database containing the user manuals, etc., about emergency measures in an emergency situation.

Meanwhile, as another embodiment, when the defibrillator storage case cover is opened, the contact sensor transmits a signal to the control unit of the defibrillator controller and the control unit notifies the server of the defibrillator management institution of opening of the defibrillator storage case cover and allows the CCTV camera attached to the inner surface of the cover to be automatically operated. At this point, when the defibrillator storage case is specifically used as a children's footrest, the case is formed to be divided into upper and lower double spaces so that the upper space is used to absorb shocks and the lower space is used to store the defibrillator. To prevent shocks such as vibration from being applied usually and to prevent malfunction, the defibrillator is configured to be removed by opening an outer cover and opening an inner cover and the common contact sensor is installed between the inner cover and the defibrillator storage case. When the cover is opened, a signal is transmitted to the control unit and the control unit determines a variation value of the signal, thereby transmitting the opening of the defibrillator storage case cover to the server of the defibrillator management institution and automatically operating the CCTV camera attached to the inner surface of the outer cover. In other words, the CCTV camera is attached to an inner surface of the outer cover.

Therefore, the server of the defibrillator management institution informs the user of the defibrillator about a method for using the defibrillator while observing situations inside the elevator with the CCTV camera attached to the defibrillator cover. Furthermore, when the defibrillator management institution wirelessly transmits the methods for using functions of specific measures depending on a patient to the defibrillator controller, the control unit loads a data for the usage method stored in the storage unit and displays it on a display, which is an output unit, to allow the user of the defibrillator to operate the relevant function.

Meanwhile, the defibrillator according to the present disclosure can be used taken to each home of the relevant line of the elevator for use in an emergency situation. At this time, when the defibrillator is separated from the defibrillator storage case, by a change of a contact switch or a weight detection switch installed in the defibrillator storage case, the control unit notifies a smartphone of an apartment manager, a security office, 119 or an emergency management department call of the emergency situation by SNS, and at the same time, a location of the relevant house number is identified through a short-range communication sensor or a GPS location sensor attached to the defibrillator, and the apartment manager takes emergency measures for a patient by communicating with the relevant house in question or visiting it in person.

The defibrillator storage case of the present disclosure may have a hexahedral shape, a cylindrical shape, or a rod shape with a fan-shaped cross section. The case is mainly made of plastic material, and the cover of the defibrillator storage case is coupled to a body of the defibrillator storage case and an inner surface thereof with a magnetic so that the cover is opened with a small force in an emergency situation and the defibrillator may be removed from the body or be operated while inside the body.

The defibrillator of the present disclosure is stored inside the defibrillator storage case and the defibrillator storage case may be installed in a wall-embedded type embedded in a wall.

Furthermore, the defibrillator storage case may be used as a box body of a footrest for children, the footrest being installed at a position of the elevator button control board or may be installed therein.

According to the present disclosure, with a regular elevator frame installed in a high-rise apartment or building and is moved up and down by carrying people or goods while double sliding doors are opened and closed:

the defibrillator storage case is firmly formed to be integrated with the elevator frame at one side surface of the elevator by welding or processing without a hole penetrating outward, and is formed to be opened inward of the elevator, and is installed close to a door so that the user can hold an opened door of the elevator with one hand and remove
the cardiac defibrillator stored in the case with the other hand.

According to the present disclosure, the elevator frame is formed with the cardiac defibrillator storage case 120 without a penetrating hole because due to a hole formed in the wall noise can enter and cause anxiety to passengers in the elevator. Furthermore, the elevator frame is formed without an inward protrusion
to prevent reduction of the internal space of the elevator

The above-described defibrillator storage case 120 integrally formed with the elevator frame of the present disclosure by welding or processing has a structure, which is firm and prevents external noise.

As the case is installed as close to the elevator door as possible, in an emergency situation such as cardiac arrest, the user can grip the opened elevator door with one hand and maintain the door in the opened state and hit and break the resin cover of the case with the other hand, thereby removing the cardiac defibrillator from the case.

The inward-opened portion of the cardiac defibrillator the defibrillator storage case 120 of the present disclosure is covered with a transparent resin cover.

Display information such as a battery condition, etc. of the cardiac defibrillator inside the case may be difficult to see, so the transparent resin cover includes a magnifier unit to make a battery condition more visible from the outside.

The transparent resin cover should be opened and closed for replacement of a battery, etc. of the stored cardiac defibrillator, and it is not preferable for the thickness to be too thick or too thin and it is not preferable for the cover to be formed to have an appropriate thickness and an appropriate material so that the cover is damaged or crushed by applying a force with one hand in an emergency situation.

In the case of cardiac arrest, follow-up measures are required, such as using a cardiac defibrillator to beat the heart within minutes after the accident, reporting to 119, etc., and transporting the patient to a hospital. In high-rise buildings, it takes several minutes for an elevator to reach to the relevant floor, so the golden time may be missed.

Therefore, it is desirable to move the elevator to the relevant floor as quickly as possible through emergency operation by contacting with a management office.

The defibrillator storage case 120 of the cardiac defibrillator of the present disclosure is firmly formed to be integrated with the elevator frame 110.

As described above, the defibrillator storage case 120 of the cardiac defibrillator of the present disclosure is sealed with respect to the external space and is opened with respect to the internal space of the elevator and has a structure protruding toward the internal space of the elevator. Therefore, the defibrillator storage case 120 cannot reduce the internal space of the elevator, the internal space being formed in accordance with space standards, and
the defibrillator storage case 120 is formed to prevent the defibrillator from hitting a passenger and the hitting between the defibrillator and a passenger occur due to due to protruding of the case.

The cardiac defibrillator storage case 120 of the cardiac defibrillator of the present disclosure is formed close to the elevator door 111,
so that in an emergency situation the user can open the elevator door with one hand and at the same time open the case cover and remove the cardiac defibrillator 100 from the internal space of the case with the other hand.

The transparent resin cover 121, which covers the opening of the cardiac defibrillator storage case 120, has the oval magnifier unit at an upper portion thereof so that the user can better observe a power condition of the cardiac defibrillator 100 inside the case in detail.

The transparent resin cover 121 may be inserted and mounted into an edge groove formed on an opening of the case, or may cover the case in a bolt-fixing manner, and it is preferable to be installed in an open and close type to replace the battery.

Considering a material, thickness, etc., the transparent resin is formed to be damaged or crushed and be opened when the user presses or hits it with force with the user's hand in an emergency situation.

Furthermore, methods for removing, using, etc., the cardiac defibrillator 100 are simply written on the transparent resin cover 121.

Meanwhile, according to the present disclosure, the defibrillator is stored inside the defibrillator storage case, and the defibrillator storage case may be provided as the wall-embedded type that is embedded in the wall as described above or may be used as a box body of a footrest for children, the footrest being provided at a position of the elevator button control board or may be installed therein.

The defibrillator storage case is manufactured into various shapes such as a hexahedral shape, a cylindrical shape, or a rod shape with a fan-shaped cross section, and the cover of the defibrillator storage case is coupled to the body and the inner surface with a magnetic so that the cover is opened with a small force in an emergency situation and the defibrillator may be removed from the body or be operated while inside the body.

Therefore, the elevator defibrillator system of the present disclosure has the remarkable effects of enabling safe use of the defibrillator and increasing the chances of saving lives by means of synchronous-control with the elevator to enable the elevator to rapidly reach the ground.

Furthermore, when the defibrillator is operated, operation thereof is automatically reported to a related institution and the related institution guides a user to perform necessary measures so that there is the remarkable effect of saving a patient by facilitating accurate operation of the defibrillator.

Furthermore, there are the effects of using the defibrillator case as a footrest, etc. and enabling convenient operation for the user to open the cover with one hand and remove the defibrillator from the case.

1. An elevator defibrillator system comprising a defibrillator (100), which is an automated cardiac defibrillator, provided inside an elevator, wherein
a controller of the defibrillator is synchronously controlled with an elevator control panel, and when the defibrillator (100) is operated, the elevator control panel stops stopping and moving functions of the elevator from and to other floors and allows the elevator to be moved only to a first floor,
a CCTV camera is provided inside the elevator or a defibrillator storage case (120), and when the defibrillator (100) is operated, a control unit transmits a signal to a server of a defibrillator management institution,
the defibrillator is stored inside the defibrillator storage case and the defibrillator storage case is used as a box body of a footrest for children, the footrest being provided at a position of an elevator button control board or is provided inside a wall, the defibrillator storage case may have a hexahedral shape, a cylindrical shape, or a rod shape with a fan-shaped cross section, and a cover of the defibrillator storage case is coupled to a body and an inner surface with a magnet so that the cover is opened in an emergency situation and the defibrillator is removed from the body or is operated while inside the case,
the defibrillator controller and the elevator control panel have respective communication modules to wirelessly communicate with each other, and the controller comprises a transmitting and receiving unit, a storage unit, and the control unit, and when the defibrillator is operated, the control unit reports an emergency situation to a smartphone of an apartment manager, a security office, 119, or an emergency management department call through the transmitting unit, and when the defibrillator is operated the control unit contacts the server of an related hospital through the transmitting unit, the hospital server contacts a smartphone of an attending specialist so that the attending specialist is on standby in a hospital emergency room, and when the defibrillator is operated, the control unit contacts the server of the related hospital through the transmitting unit, and the hospital server contacts a smartphone of a driver of an ambulance, so that the ambulance will arrive at a building with the relevant elevator and be on standby,
the CCTV camera is installed inside the elevator or the defibrillator storage case, and the camera inside the case is attached to an inner surface of the cover to enable a user to easily face the camera when the inner surface of the cover is positioned to face the user, so when the defibrillator is operated, the control unit transmits a signal to the server of f the defibrillator management institution, and when the defibrillator is operated and a defibrillator operation signal is received, a server control unit of a public health center or a disease control and prevention center, which is the defibrillator management institution, operates speakers provided inside the defibrillator case to allow the user to manually turn on the CCTV camera by voice,
when the cover of the defibrillator storage case is opened, a contact sensor transmits a signal to the control unit of the defibrillator controller, and the control unit transmits opening of the defibrillator storage case cover to the server of the defibrillator management institution and allows the CCTV camera attached to the inner surface of the cover to be automatically operated, and
when the defibrillator storage case cover is opened, a signal is transmitted to the control unit, and the control unit determines a variation value of the signal and transmits the opening of the defibrillator storage case cover to the server of the defibrillator management institution and allows the CCTV camera attached to the inner surface of an outer cover to be automatically operated, and the CCTV camera is attached to the inner surface of the outer cover, and the server of the defibrillator management institution informs the user of the defibrillator about a method for using the defibrillator while observing internal situations of the elevator with the CCTV camera attached to the defibrillator storage case cover, and when the defibrillator management institution wirelessly transmits methods for using functions of specific measures depending on a patient to the defibrillator controller, the control unit loads a data for the usage method stored in the storage unit and displays the data on a display, the display being an output unit, to allow the user of the defibrillator to operate the relevant function.
